(19) United States
(12) Patent Application Publication
(10) Pub. No.: US 2006/0062827 A1
(43) Pub. Date: Mar. 23, 2006

(54) Nutritional supplement composition and method

(75) Inventors: Minter H. Dopson, Warrenville, SC (US); Joseph H. Davis, West Columbia, SC (US); Robin P. Gunwall, Lexington, SC (US)

Correspondence Address: NELSON MULLINS RILEY & SCARBOROUGH, LLP, 1320 MAIN STREET, 17TH FLOOR, COLUMBIA, SC, 29201, US

(73) Assignee: Gel Dynamics, LLC

(21) Appl. No.: 10/324,637

(22) Filed: Dec. 20, 2002

(51) Int. Cl. A61K 47/00

(52) U.S. Cl. 424/439

(57) ABSTRACT

A nutritional supplement composition containing a mixture of whey protein, amino acids, and other beneficial constituents, and a method for making the composition in a nutritionally compact, easy-to-use form. The composition is made by processing a mixture of whey protein, amino acids, minerals, vitamins, and other selected ingredients at an appropriate temperature and pH. By adjusting the ph of the mixture to within the approximately range of 2.5-5.0, and by processing at an appropriate temperature, it is possible to obtain a gel composition that contains a surprisingly large fraction of whey proteins (and other beneficial constituents) in readily bioavailable form. The composition can be packaged into conveniently-sized containers that are as easy to store or transport as they are easy to use.

### BACKGROUND OF THE INVENTION

1. Field of the Invention

The present invention relates to a nutritional supplement composition. In particular, the present invention relates to a nutritional supplement composition containing whey protein, amino acids and other beneficial ingredients in bioavailable form, and to a method for making the composition. The present invention also relates to food products that contain the composition, and to methods for using the composition.

2. Discussion of Background

Throughout recorded history, good nutrition has been recognized as one of the most important factors in maintaining good health. Protein in particular is recognized as an important nutrient, whether obtained from meat, dairy products, or other sources. Dairy products are widely used as part of a healthy diet: indeed, the consumption of fermented whole milk (yogurt) is may be associated with unusually long life expectancy in the parts of the former Soviet Union, particularly the Caucasus region.

Whey, also known as Serum lactis, is one of the by-products of the dairy industry. It is produced by acidifying milk at a pH near the isoelectric point of the casein, then separating the curd to recover the whey liquid. Alternative methods include flocculating or coagulating the milk caseins by adding rennet to whole milk, then separating out the whey liquid. So-called “sweet whey” is produced by flocculation at a pH that is approximately that of milk, but above about 5.8-6.0. Typically, sweet wheys are obtained from cooked or uncooked pressed curds such as those used in making Emmental, Gruyere, and Cheddar-type cheese. Acid wheys result from the processing of fresh curds, and “mixed” wheys result from the processing of soft curds and marbled curds (i.e., curds used to make blue cheese).

The composition of whey varies depending on the composition of the starting milk and the selection of process. In general, whey contains fats, minerals, lactic acid, coagulated enzymes, and nitrogenous compounds, but lacks most of the fat, fat-soluble vitamins, caseins, and casein-bound calcium and phosphate found in whole milk or skim milk. For example, approximately 80% of the total protein content of cows milk, but only about 20% of the protein content of whey, consists of caseins. The protein fraction of whey includes a variety of soluble milk proteins, b-lactoglobulins, a-lactoalbumins, serum albumins, immunoglobulins, and amino acids.

Whey has traditionally been used as a fertilizer and as a constituent of animal feed; it is also a widely-used and well-documented folk remedy. Jaundice, skin infections, genito-urinary tract infections, venereal diseases, epilepsy, and fever have been treated with whey. More recently, it has been determined that regular consumption of whey results in an immuno-enhancing effect, and may contribute to detoxification of cancer-causing environmental agents. Whey is also an important dietary supplement, used by athletes, bodybuilders, and others interested in achieving—and maintaining—optimum nutritional status, immune system status, and general health. Conditioning programs frequently include the consumption of large quantities of liquid whey several times daily. Thus, there is a large group of consumers who are interested in convenient dietary sources of whey.

A wide variety of nutritional supplements that contain whey, amino acids, and other beneficial ingredients are available to consumers (for purposes of this specification, the terms “nutritional supplement,” “food supplement,” and “dietary supplement” are used interchangeably). By way of example, Almada, et al. (U.S. Pat. No. 5,726,146) disclose a non-steroidal anabolic dietary supplement and method for increasing lean body mass without increasing fat mass. The supplement includes creatine, taurine, ribonucleic acid, and a carbohydrate; it may include other ingredients such as alpha-ketoglutaric acid, beta-hydroxy-beta-methyl butyric acid, and salts thereof. It may be consumed alone or combined with a nutrient base which includes proteins, carbohydrates, vitamins, minerals, and other amino acids.

Bounous, et al. (U.S. Pat. No. 5,451,412) and Gold, et al.(U.S. Pat. No. 5,230,902) provide undenatured whey protein concentrates used as food supplements, and methods of making the concentrates. The concentrates contain proteins in an “essentially” undenatured state; their biological activity depends on the overall amino acid and small peptides pattern resulting from the protein components. Vitamins B1 and B2 may be added to further increase biological activity.

Henderson (PCT Application No. WO00/37087) discloses a comprehensive dietary supplement containing bioavailable minerals, vitamins, phytonutrients, herbs, antioxidants, and enzymes. The minerals are in the form of amino acid chelates.

Jarowski (U.S. Pat. No. 5,559,142) shows a universal dietary protein supplement which consists of a blend of four essential amino acids (L-Tryptophan, L-Methionine, L-Valine, L-Lysine Monohydrochloride). The relative proportions of at least the first three amino acids conform to the respective proportions found in human blood plasma (after fasting).

Maubois, et al. (U.S. Pat. No. 4,427,658) disclose a total enzymatic hydrolysate derived from whey proteins and process for obtaining the hydrolysate. Their composition includes a peptidic hydrolysate with substantially no residual proteins, where at least 50% of the peptides contain 2-5 amino acids and 70%-90% of the nitrogen present in the peptides has less than 10 amino acids. The composition is used in as a food supplement (in the form of pills), diet food, or intensive care food (administered via the enteral tract).

Meade (U.S. Pat. No. 5,631,031) describes a water-insoluble amino acid salt which can be used as a supplement for ruminant animals and humans. The supplement can be incorporated into flour or other food-compatible carriers.

O'Donnell, et al. (European Patent No. 1 108 429 A2) provide an amino acid composition for use as an amino acid or protein supplement, aimed at the treatment and/or management of diseases of amino acid metabolism such as phenylketonuria. The composition includes a selection of amino acids, other standard nutrients (minerals, trace elements, vitamins, flavorings, etc), and is combined with a thickening agent such as pre-gelatinized starch, modified starch, gum, or cellulose.

Regnault, et al. (European Patent No. 1 208 749 A1 and PCT Application No. WO99/5517) describe a protein supplement, methods for preparing and using the supplement, and food compositions that contain the supplement. The supplement contains at least 25% (preferably 80%) protein by weight; the tryptophan transfer/neutral amino acid ratio is at least 0.06.

Shay, et al. (U.S. Pat. No. 4,439,525) disclose high methionine content yeasts of the strain Pichia pastoris. Their yeasts have good productivity in single cell protein production, and also have a higher amino acid content than other yeasts. The yeast cells are dried for use; the fermentor effluent can also be dried for use as a high-protein animal feed.

Whey is also used as a fertilizer. For example, Brinker, et al. (U.S. Pat. No. 6,245,713) describe treatment compositions having enhanced biological effectiveness for application to the foliage of a plant for enhancing the efficiency of delivery to foliar-applied substances to their sites of action. The compositions include an herbicide, an alkylether surfactant, amine surfactants, and an amino group that is cationic or that can be protonated to become cationic. They are provided in solid or liquid concentrate form, and are mixed with water prior to use.

Kinnersley, et al. (U.S. Pat. No. 5,840,656 and U.S. Pat. No. 5,350,735) disclose water-soluble compositions, and methods for utilizing the compositions to enhance fertilizer uptake and promote plant growth. The compositions include water-soluble poly(organic acids), particularly poly(amino acids) such as poly(aspartic acid) and their copolymers, and can be applied by spraying.

Miyazawa, et al. (European Patent No. 1 208 749 A1 and U.S. Application No. 20020095696) describe a tillering promoter that is applied to the leaf surfaces of plants to promote the production of side shoots by the plant. Their composition includes amino acids such as proline or inosine as the effective ingredients. U.S. Pat. No. 6,448,202 describes a withering-preventing agent that contains amino acids.

Despite the many different products that are available, no known product contains an effective amount of whey in a convenient, easy-to-use, easy-to-carry, and easy-to-store form. As noted above, bodybuilders and other athletes may consume several pints of liquid whey daily, preferably every 2-6 hours or thereabouts to maintain an even body load. Whey in liquid or solid form is readily available at health food stores, but is essentially impossible to find at the average restaurant or supermarket. Therefore, the average user who wishes to maintain a desired consumption schedule must carry his or her own supply to work or school. Liquid (i.e. bottled) whey is bulky and inconvenient to carry. Dehydrated, solid whey must be reconstituted in a blender, a common kitchen appliance which nevertheless is rarely found in the typical workplace. There remains a need for a nutritional composition that contains whey in a bioavailable form, that is cost-effective to manufacture, and that is easy and convenient to use.

### SUMMARY OF THE INVENTION

According to its major aspects and broadly stated, the present invention includes a nutritional supplement composition containing a mixture of whey protein, amino acids, and other beneficial constituents, and a method for making the composition in a nutritionally compact, easy-to-use form. The composition can be packaged into conveniently-sized containers that are as easy to store or transport as they are easy to use, and are shelf-stable without requiring refrigeration.

An important feature of the present invention is the method for making the composition, which includes the step of processing a mixture of whey protein, amino acids, minerals, vitamins, and other selected ingredients at an appropriate temperature and pH. By adjusting the ph of the mixture to within the approximately range of 2.5-5.0, and by processing at an appropriate temperature, it is possible to obtain a gel composition that contains a surprisingly large fraction of whey proteins (and other beneficial constituents) in readily bioavailable form.

Another feature of the present invention is the composition itself As noted above, the composition contains a relatively large amount of whey (at least approximately 0.1 gram whey per mL water, preferably approximately gram whey per mL water) as well as amino acids (preferably low molecular weight amino acids), low-glycemic-index sugar, minerals (magnesium, calcium, zinc, manganese, chromium, selenium, molybdenum, potassium, boron, etc.), nutritionals (MSM, creatine, L-glutamine, taurine, malic acid, vitamins and vitamin precursors (vitamin A, carotene, cryptoxanthin, retinol, 3-dehydroretinol, vitamin C (ascorbic acid), vitamin E (tocopherol), etc.)), beneficial enzymes, and homeopathics. The composition may also include additional sources of protein, including but not limited to egg protein and soy protein, as well as coloring and flavoring agents, fragrances, and other ingredients known and used in the art.

Still another feature of the present invention is the use of whey solids, which are an important constituent in many nutritional programs. Essentially any whey solids may be used with the invention, of molecular weights ranging between approximately 500 and 50,000, including mixed wheys resulting from the mixing of byproducts from the manufacture of different types of cheeses and whey solids prepared from raw or heat-treated milk by lactic or rennet precipitation followed by dehydration or evaporation.

Yet another feature of the present invention is the low-glycemic-index sugar, preferably Kiwi sugar or the like, which helps optimize metabolizing of the composition and helps avoid the possibly-deleterious effects of a sugar “high” which can result from over-consumption of sucrose. The use of low-glycemic-index sugar also makes the composition suitable for use by diabetics and other who must control their intake of dietary sugars. Other suitable sugars include, but are not limited to, stevia, saccharin, sucrolose, NUTRASWEET, sucrose, and fructose.

Another feature of the present invention is the packaging. The end product composition is preferably packaged in containers made of moisture-barrier material, such as food-grade thermoplastic polymers, metal foil, and combinations thereof The product is shelf-stable, does not require refrigeration or other special storage conditions, and can be opened and used “as is.” A single container may contain one or more servings of the composition: for example, containers with resealable bead-and-groove closures or other recloseable or resealable closures permit the end user to consume just as much of the composition as he or she wishes, and save the rest for later use. Single-serving containers are convenient for packing with a “brown bag” lunch.

Still another feature of the present invention is the ability to use the composition in a variety of edible products, including but not necessarily limited to nutraceuticals, dietary supplements, and pharmaceutical or veterinary compositions.

Yet another feature of the present invention is the use of the composition, and products containing the composition, as part of an overall health and fitness program. The composition is broadly useful as a nutritional supplement. Regular consumption of the composition (or products containing the composition) as part of a diet and exercise program helps the user build and maintain muscle mass as well as enhancing overall health and well-being. The composition is also believed to help enhance the user's immune system when consumed on a regular basis.

Other features and advantages of the present invention will be apparent to those skilled in the art from a careful reading of the Detailed Description of Preferred Embodiments presented below and accompanied by the drawings.

### BRIEF DESCRIPTION OF THE DRAWINGS

In the drawings,

FIG. 1 is a flow chart illustrating a method for making a composition according to a preferred embodiment of the present invention.

### DETAILED DESCRIPTION OF PREFERRED EMBODIMENTS

In the following detailed description of the invention, the drawing is intended to be read together with the specification, and is to be considered a portion of the entire written description of this invention as required by 35 U.S.C. § 112.

The present invention is based on the discovery that a suitable mixture of whey protein, amino acids, and other beneficial constituents can be processed to form a gel composition. By processing the mixture at an appropriate temperature and pH, it is possible to obtain a gel composition that contains a surprisingly large fraction of whey proteins (and other beneficial constituents) in readily bioavailable form. The composition can be packaged into conveniently-sized containers that are as easy to store or transport as they are easy to use.

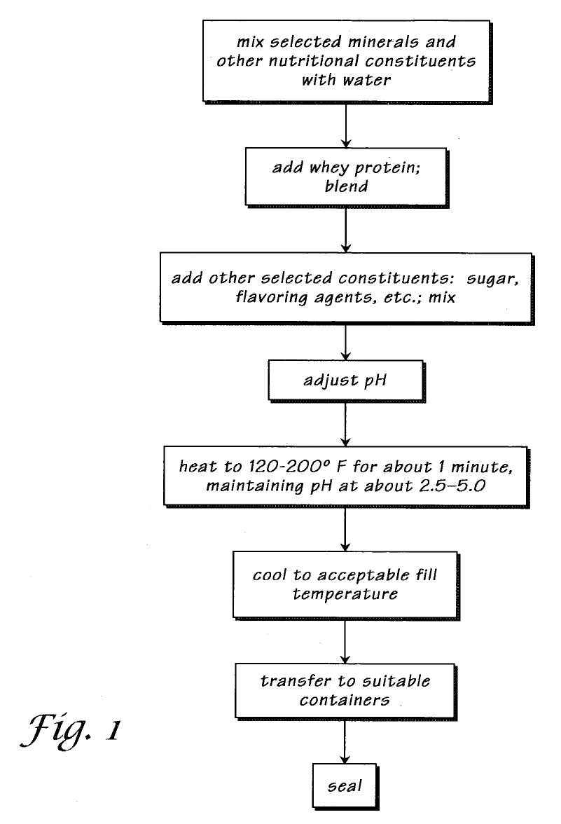

In accordance with a preferred embodiment of the invention, a nutritional composition containing whey is manufactured generally as shown in FIG. 1. The method for making the composition includes the following steps:

1. Add a selected quantity of minerals, malic acid, amino acids (preferably low molecular weight amino acids), and other nutritional constituents to water, and blend.

Suitable minerals include, but are not limited to, magnesium, calcium, zinc, manganese, chromium, selenium, molybdenum, potassium, boron, and mixtures thereof Nutritional constituents that may be added to the composition include MSM, creatine, L-glutamine, taurine, malic acid, vitamins and vitamin precursors (vitamin A, carotene, cryptoxanthin, retinol, 3-dehydroretinol, vitamin C (ascorbic acid), vitamin E (tocopherol), etc.), beneficial enzymes, homeopathics, and flavoring agents. The composition may also include additional sources of protein, including but not limited to egg protein and soy protein.

2. Add a selected quantity of whey protein, in solid form, to the water/mineral/nutritional blend; homogenize.

Essentially any whey solids may be used with the invention, of molecular weights ranging between approximately 500 and 50,000. For example, mixed wheys resulting from the mixing of byproducts from the manufacture of different types of cheeses may be used with the invention. Alternatively, whey solids prepared from raw or heat-treated milk by lactic or rennet precipitation followed by dehydration or evaporation may also be useful. Preferably, the whey solids have been purified by ultrafiltration, microfiltration, diafiltration, ion exchange chromatography, or other suitable process prior to dehydration.

The ratio of whey solids to water may vary: ratios between approximately 0.1-2 grams whey solids per mL of water are broadly suitable for the practice of the present invention. More preferably, the ratio is approximately 0.5-1.5 grams whey solids per mL of water, and most preferably, approximately 0.8-1.2 grams whey solids per mL of water. The optimum whey: water ratio depends on the other constituents of the composition and the nature of those constituents, as well as the desired end product.

3. Add a suitable quantity of sugar to the mixture.

Suitable sugars include, but are not limited to, low-glycemic-index sugars, Kiwi sugar, stevia, sucrolose, saccharin, NUTRASWEET, fructose, glucose, and sucrose. Low-glycemic-index sugars are particularly useful, since these types of sugars do not produce as elevated blood sugar levels after consumption as other types of sugars. While many factors influence an individual's blood sugar response to foods (including sugars), diabetics and those with impaired insulin response may benefit from consuming foods of relatively low glycemic index. Incorporation of low glycemic index foods in the diet is associated with reduced blood glucose, insulin, and lipid levels, as well as increased colonic fermentation, increased bacterial urea utilization, and increased production and absorption of short chain fatty acids in the colon.

4. If desired, add additional constituents, including but not necessarily limited to coloring agents, flavoring agents (vanilla, chocolate, strawberry, kiwi, orange, lemon, malt, etc.), fragrances, immune-system enhancers, antioxidants, fillers, stabilizers, antimicrobials, preservatives, pH-balancing agents, and combinations thereof.

Each ingredient of the composition is present in an amount that, as a percentage of the total weight of the composition, is effective either alone or synergistically with the other constituents to achieve the desired results. A composition according to the invention may contain approximately 5-50 wt. % whey (as added in Step 2 above), with the balance consisting of other constituents and water. Constituents such as minerals, vitamins, flavoring agents, and so forth may constitute 1-50 wt. % of the composition. However, amounts outside these range may also be useful.

5. Adjust the pH of the mixture to approximately 2.5-5.0, preferably to approximately 4.3, by adding a malic acid or other suitable acid. The composition is naturally pH-balanced when formulated with selected ingredients as described below. However, pH-balancing agents may be added if needed.

6. Pasteurize the pH-adjusted mixture.

Pasteurization can be carried out as a batch process, wherein the temperature of the mixture is raised to between approximately 120-200° F. for approximately one minute. More preferably, pasteurization is carried out at a temperature between approximately 160-180° F.; most preferably, at a temperature of approximately 170° F.

Alternatively, the mixture is processed by an in-line pasteurization system that elevates its temperature to the selected level for approximately one minute dwell time, then cools the mixture to an acceptable fill temperature.

Irradiation, cold-sterilization, and/or other suitable processing techniques may be used instead of (or in addition to) pasteurization, providing that the composition contains a suitable amount of at least one food-grade constituent having preservative action. For example, grapefruit seed extract and other fruit-derived extracts may be useful.

7. After the mixture has cooled to an acceptable fill temperature, pump into suitable containers such as pouches, gel-packs, and so forth. Seal the filled containers.

The mixture may be packaged in any suitable containers, preferably sterilizable containers made of moisture-barrier material. Suitable materials include food-grade thermoplastic polymers such as polystyrene, polyester, low density polyethylene, metal foil, and combinations thereof. For example, a suitable container might be a heat-sealable bag with walls that need be no more than 0.05-0.25 mm thick. Each container may contain one or more servings of the composition, as may be preferred. For example, a single-serving container might contain 1-2 ounces of the product, and measure no more than approximately 1½ by 4 inches (that is, about the size of typical granola bars, snack bars, and the like). Containers with resealable bead-and-groove closures or other recloseable or resealable closures permit the end user to consume just as much of the composition as he or she wishes, and save the rest for later use.

The method as described above results in a gel-type product that contains a surprisingly large amount of whey protein in an easy-to-use, easy-to digest form. Instead of having to mix and drink large quantities of reconstituted liquid whey, a health-conscious consumer needs only to open a container of the composition and eat the contents. The product is shelf-stable, requiring no refrigeration or other special storage to maintain quality. The containers can be opened and used ad libitum, without the need to heat, cool, reconstitute, or otherwise prepare the product for consumption.

The present invention is further illustrated by the following non-limiting example.

### EXAMPLE

A mixture of minerals (magnesium, calcium, zinc, manganese, chromium, selenium, molybdenum, potassium, and boron), amino acids, malic acid, and water was prepared. Whey protein solids, in an amount of 1 gram per 1 mL, were blended into the mixture. Kiwi sugar was added to taste. Additional constituents (coloring agents, preservatives) in an amount less than 2 wt. % of the composition were added, and the pH was adjusted to approximately 4.3. The resulting mixture was heated to a temperature of 170° F. for 1 minute, cooled, transferred to flexible, heat-sealable containers, each container holding about 2 ounces of product.

The product had a gel-like consistency, and was determined to have an acceptable mouth “feel” and acceptable flavor as well as being more convenient than whey solids or reconstituted whey liquid.

A composition according to the present invention may be used as a nutritional supplement by athletes, bodybuilders, and other interested in achieving and maintaining optimum nutritional status. If desired, the composition can be incorporated into other edible products, including nutraceuticals, and pharmaceutical or veterinary compositions. It may also be administered parenterally or by mouth as a component of a therapeutic nutritional program or for patients in intensive care, nursing home patients, and those with absorption problems. It may also be useful as a dietary supplement for children, including children who are reluctant to consume conventional vitamin supplements or who are “picky eaters.” The composition may also be useful as a general immune system toner or optimizer, since ingestion of whey is believed to enhance the immune system.

With respect to the above description of the invention, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

Therefore, the foregoing description is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention. Thus, it will be apparent to those skilled in the art that many changes and substitutions can be made to the preferred embodiment herein described without departing from the spirit and scope of the present invention as defined by the appended claims.

1-8. (canceled)

9. A method for making a nutritional composition, said method comprising the steps of:
(a) mixing a quantity of minerals with water to form a first mixture;
(b) mixing approximately 0.13-2.0 grams of whey protein solids per mL of water in said first mixture to form a second mixture;
(c) adding at least one sugar to said second mixture to form a third mixture; and
(d) adjusting the pH of said third mixture to between 2.5 and 5.0.

10. The method as recited in claim 9, further comprising the step of adding to said second mixture at least one ingredient selected from the group consisting of minerals, vitamins, vitamin precursors, beneficial enzymes, homeopathics, egg protein, soy protein, and creatine.

11. The method as recited in claim 9, further comprising the step of adding to said second mixture at least one ingredient selected from the group consisting of flavoring agents, coloring agents, fragrances, preservatives, and antimicrobials.

12. The method as recited in claim 9, further comprising adding a quantity of malic acid to said first mixture.

13. The method as recited in claim 9, wherein step (d) further comprises adding a suitable quantity of malic acid to said second mixture.

14. The method as recited in claim 9, wherein step (e) further comprises heating said pH-adjusted mixture to a temperature between approximately 160° F. and 180° F.

15. The method as recited in claim 9, wherein step b further comprises mixing approximately 0.8-1.2 grams of whey protein solids per mL of water to form said second mixture.

16. The method as recited in claim 9, wherein step (c) further comprises adding a low-glycemic-index sugar, kiwi sugar, or stevia to form said third mixture.

17-20. (canceled)
